(19) United States
(12) Patent Application Publication
(10) Pub. No.: US 2021/0008530 A1
(43) Pub. Date: Jan. 14, 2021

(54) CATALYSTS AND METHODS FOR PRODUCING ACETIC ACID FROM METHANE, CARBON MONOXIDE, AND OXYGEN

(71) Applicants: University of Kansas, Lawrence, KS (US); University of Notre Dame Du Lac, Notre Dame, IN (US)

(72) Inventors: Franklin Tao, Lawrence, KS (US); Weixin Huang, Menlo Park, CA (US); Yu Tang, Lawrence, KS (US); Yuting Li, Lawrence, KS (US)

(21) Appl. No.: 17/040,707

(22) PCT Filed: Mar. 26, 2019
(86) PCT No.: PCT/US2019/024013

§ 371 Date: Sep. 23, 2020

Related U.S. Application Data
(60) Provisional application No. 62/648,105, filed on Mar. 26, 2018.

(51) Int. Cl. B01J 29/068; B01J 29/44; B01J 35/00; C07C 29/52; C07C 51/10

(52) U.S. Cl. CPC B01J 29/068; B01J 29/44; B01J 2229/186; C07C 29/52; C07C 51/10; B01J 35/002

(57) ABSTRACT

Catalysts for producing one or more oxygenated products from methane are provided. In embodiments, the catalyst comprises active sites comprising isolated, cationic transition metal M′ atoms covalently bound to internal surfaces of pores of a porous metal M″ silicate, wherein M′ is Rh or Ir, and further wherein the M′ atoms are bound to five oxygen (O) atoms. Methods for making and using the catalysts are also provided.

### CROSS-REFERENCE TO RELATED APPLICATIONS

The present application claims priority to U.S. Provisional Patent Application No. 62/648,105 that was filed Mar. 26, 2018, the entire contents of which are hereby incorporated by reference.

### REFERENCE TO GOVERNMENT RIGHTS

This invention was made with government support under DE-SC0014561 awarded by the Department of Energy. The government has certain rights in the invention.

### BACKGROUND

CH₄ has been one of the inexpensive energy resources since the maturation of hydraulic fracturing technology. So far, most processes of transformation of CH₄ to intermediate compounds for chemical industries including steam or dry reforming, partial oxidation, and oxidative coupling are performed at high temperatures. One side effect of these processes is the deactivation of catalysts due to coke formation. Another is the input of a huge amount of energy since they are performed at high temperatures. Thus, activation of C—H of CH₄ at a low temperature is necessary in order to transform shale gas to intermediate compounds of chemical industries in an energy-efficient manner.

Acetic acid is one of the important intermediates of chemical industries. The global demand is 6.5 million metric tons per year (Mt/a). Currently, it is produced from methanol carbonylation, in which CO reacts with methanol to form acetic acid. However, methanol is synthesized from CO and H₂, which are produced from steam reforming processes of either methane or coal at high temperatures. Replacement of the current high temperature catalysis toward production of acetic acid with catalysis at low temperatures would be feasible if a catalytic process on a heterogeneous catalyst could efficiently directly transform CH₄ to acetic acid under a mild condition.

### SUMMARY

Provided are catalysts for producing acetic acid from methane, carbon monoxide, and oxygen. Methods for making and using the catalysts are also provided.

Catalysts for producing one or more oxygenated products from methane are provided. In embodiments, the catalyst comprises active sites comprising isolated, cationic transition metal M′ atoms covalently bound to internal surfaces of pores of a porous metal M″ silicate, wherein M′ is Rh or Ir, and further wherein the M′ atoms are bound to five oxygen (O) atoms. Methods for making and using the catalysts are also provided.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A-1H relate to the structural characterization of isolated, cationic rhodium atoms in ZSM-5. FIG. 1A shows a TEM image of particles of 0.10 wt % Rh/ZSM-5; scale bar: 10 nm. FIG. 1B shows a TEM image of particles of 0.50 wt % Rh/ZSM-5; scale bar: 10 nm. FIG. 1C shows the Rh 3 d XPS peak of 0.10 wt % Rh/ZSM-5 and 0.50 wt % Rh/ZSM-5. FIG. 1D shows the energy space of Rh K-edge of 0.10 wt % Rh/ZSM-5 and Rh foil (reference sample) of X-ray absorption spectra. FIG. 1E shows the r-space of Rh—K edge of experimental and calculated data of the k2-weighted Rh K-edge EXAFS spectra of used 0.10 wt % Rh/ZSM-5. FIG. 1F lists the coordination number and bond length on average of the used 0.10 wt % Rh/ZSM-5. FIG. 1G shows the r-space of Rh—K edge of experimental and calculated data of the k2-weighted Rh K-edge EXAFS spectra of Rh metal foil. FIG. 1H shows the r-space of Rh—K edge of experimental and calculated data of the k2-weighted Rh K-edge EXAFS spectra of Rh₂O₃ nanoparticles supported on Al₂O₃. FIG. 11 illustrates the structure of an active site of the catalyst, including the isolated, cationic Rh atom bound to 5 oxygen atoms and anchored in a pore of ZMS-5.

FIG. 2 shows the catalytic performance of 0.10 wt % Rh/ZSM-5. Yields of acetic acid, formic acid, and methanol as well as the selectivity to acetic acid as a function of different pressures of CH₄ (10 or 50 bars) and different reaction times (2 hr or 12 hrs). 28 mg of 0.10 wt % Rh/ZSM-5 was used for each catalysis. Each catalysis condition used 10 bar CO and 8 bar O₂ and certain pressure of CH₄ as noted on X-axis (10 or 50 bars). In each case, the catalysis temperature was 150° C.

FIGS. 3A-3C show the influence of partial pressure of O₂, CO and CH₄ on catalytic performances. Yields of methanol (left), formic acid (middle) and acetic acid (right) in the chemical transformation of CH₄ at 150° C. in aqueous solutions at different pressures of O₂, CO, CH₄. FIG. 3A shows the results 35 bar CH₄, 10 bar CO and different pressures of O₂ at 150° C. for 2 hrs. FIG. 3B shows the results at 50 bar CH₄, 8 bar O₂ and different pressures of CO at 150° C. for 1.5 hrs. FIG. 3C shows the results at 10 bar CO, 8 bar O₂ and different pressures of CH₄ at 150° C. for 2 hrs.

FIGS. 4A-4D show ¹³C NMR studies using ¹³CO and ¹³CH₄. ¹³C NMR spectra of products of acetic acid, formic acid and methanol on 28 mg 0.10 wt % Rh/ZSM-5 at 170° C. for 10 hrs in gas of (FIG. 4A) mixture of 0.7 bar ¹³CO, 6.3 bar CO, 14 bar CH₄ and 8 bar O₂, (FIG. 4B) mixture of 7 bar CO, 14 bar CH₄ and 8 bar O₂, (FIG. 4C) mixture of 7 bar CO, 0.7 bar ¹³CH₄, 13.3 bar CH₄ and 8 bar O₂, and (FIG. 4D) mixture of 7 bar CO, 14 bar CH₄ and 8 bar O₂. FIGS. 4A and 4B are isotope experiments; FIGS. 4C and 4D are their corresponding contrast experiments.

FIGS. 5A-5D show isotope studies for elucidating whether acetic acid could be formed through coupling methanol with CO. FIG. 5A shows two potential pathways α and β for production of acetic acid; in pathway α, CH₃OH is not an intermediate for formation of CH₃COOH; in pathway β, CH₃OH is an intermediate for formation of CH₃COOH. FIG. 5B shows potential catalytic products formed from 0.10 wt % Rh/ZSM-5 in the mixture of ¹³CH₃OH and H₂O in solution under mixture of CH₄, CO and O₂ if the transformation of CH₄, CO and O₂ follows pathway α, β, or both α and β. FIG. 5C shows the NMR spectrum of the products formed from 28 mg of 0.10 wt % Rh/ZSM-5 after reaction in 10 bar CH₄, 5 bar CO, and 4 bar O₂ at 150° C. for 1 h; there was not any isotope-labelled methanol, ¹³CH₃OH added to the rector before catalysis. FIG. 5D shows the NMR spectrum of the products formed from 28 mg of 0.10 wt % Rh/ZSM-5 after reaction in 10 bar CH₄, 5 bar CO, and 4 bar O₂ at 150° C. for 1 h.; notably, 1.0 mmol ¹³CH₃OH was added to H₂O before catalysis.

FIGS. 6A-6B show the computational studies showing the minimum-energy paths and reaction schematic for acetic acid formation from CH₄, CO, and O₂ on Rh₁O₅/ZSM5. The formation of acetic acid on the Rh₁O₅ site is illustrated in a catalytic cycle starting with the singly dispersed Rh₁O₅ site. The balanced reaction cycle consumes one O₂, two CH₄ and two CO to make two CHCOOH molecules (2CH₄+2CO+O₂=2CH₃COOH). FIG. 6A shows the structures of intermediates and transition states for a complete catalytic cycle. FIG. 6B shows the energy profile for transforming CH₄, CO and O₂ to CH₃COOH. Transition states are highlighted with the double dagger symbol.

### DETAILED DESCRIPTION

Provided are catalysts for producing oxygenated products from methane, carbon monoxide, and oxygen. Methods for making and using the catalysts are also provided. The catalysts comprise active sites comprising single (i.e., isolated) transition metal cations covalently bound to internal surfaces of pores of porous silicate supports. The active sites have a unique structure which is achieved using a unique processing method. It is believed that this unique structure enables the direct conversion of methane via the concerted coupling of methane, carbon monoxide and oxygen to produce oxygenated products (including acetic acid, formic acid and methanol) with high activity and selectivity, even under mild conditions, e.g., 150° C.

In one aspect, the catalysts are provided. In embodiments, a catalyst comprises active sites comprising isolated, cationic transition metal (M′) atoms covalently bound to internal surfaces of pores of a porous metal (M″) silicate support. The label M′ designates the transition metal. In embodiments, M′ may be Rh (rhodium) or Ir (iridium). The label M″ designates the metal of the porous metal silicate support, which may be Al (aluminum). In embodiments, the porous metal silicate support is microporous aluminosilicate, e.g., zeolite. An illustrative zeolite is ZSM-5. Such porous metal silicate supports comprise metal-oxygen (M″-O) and silicon-oxygen (Si—O) bonds throughout the lattice structure of the solid material.

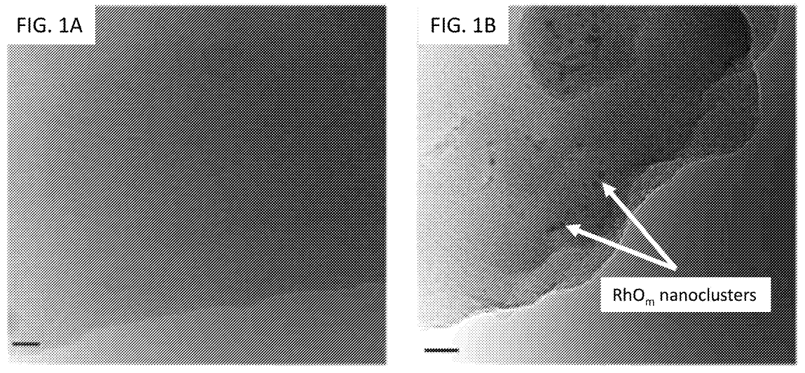

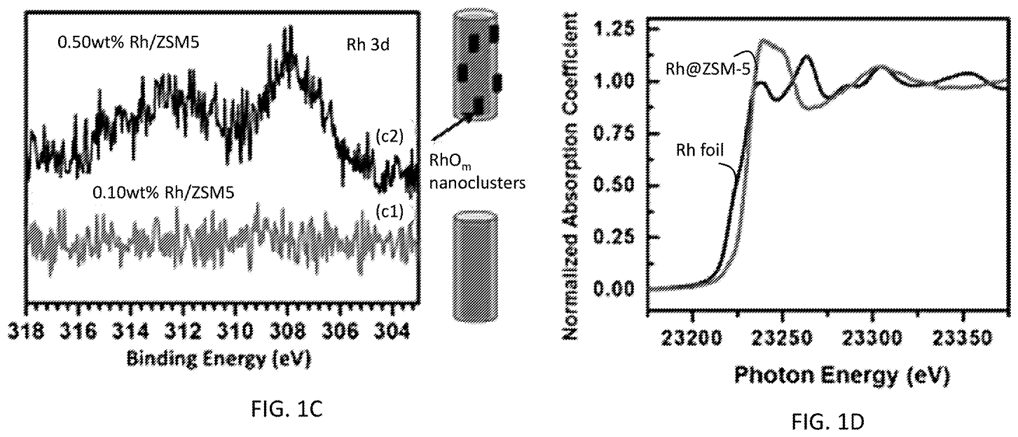

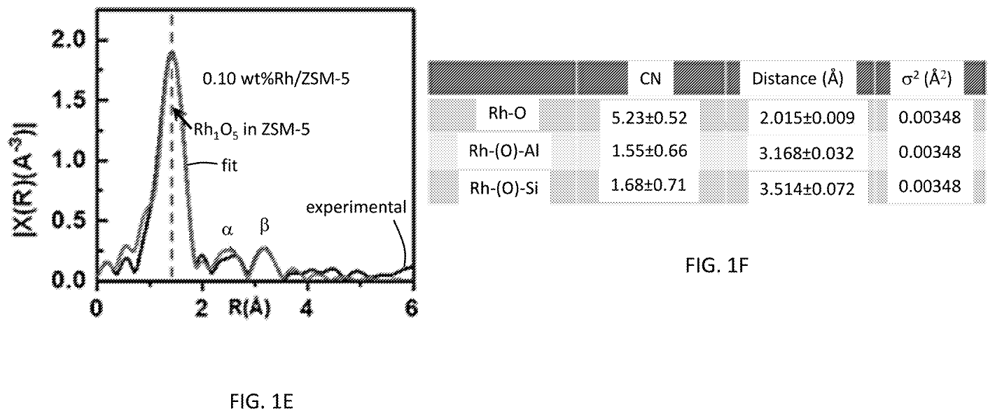

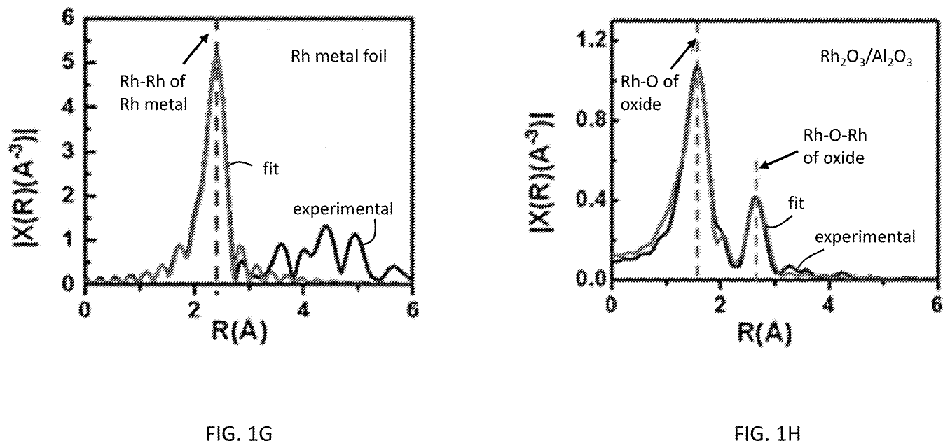

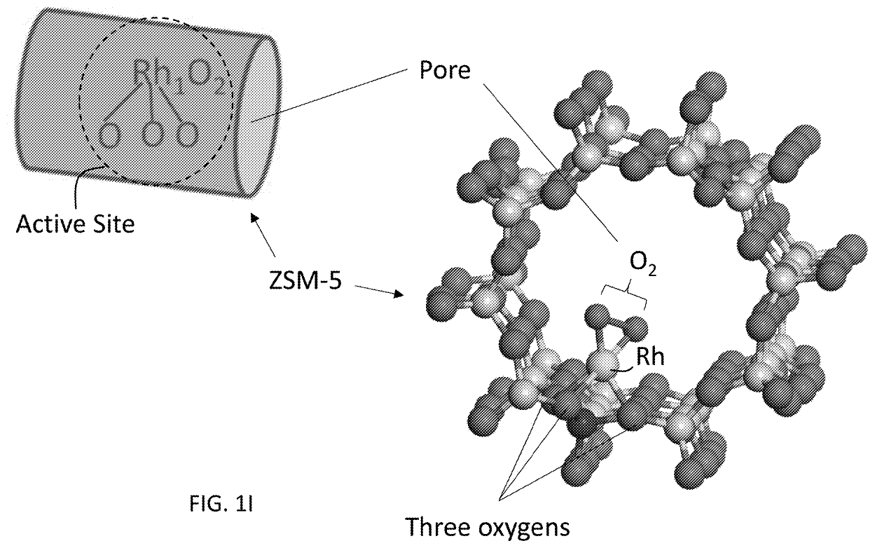

As noted above, the active sites comprising the isolated, cationic transition metal (M′) atoms have a unique structure as characterized by chemical environment surrounding each isolated, cationic M′ atom. The chemical environment refers to the coordination number (CN) of the M′ to oxygen (O) atoms as well as the bonding of the O atoms to other species. The isolated, cationic transition metal M′ atoms may be covalently bound to five oxygen (O) atoms. Such catalysts may be designated by the formula, (M′)₁-O₅. The number of M′-O bonds, i.e., the CN, may be determined using extended X-ray absorption fine structure spectroscopy (EXAFS) as described in the Example, below. (See also FIG. 1F.) In embodiments, three of these five oxygen atoms are those which are also covalently bound within the lattice structure of the porous metal M″ silicate support. In these embodiments, the other two of the five oxygen atoms are bound to one another (in addition to M′). That is, in embodiments, each active site has the structure shown in FIG. 1I. This active site is hereby designated as (O₂)=M′≡(O)₃. In this structure, the oxygen atoms of O₂ are bound to one another, effectively forming an oxygen bridge over the isolated, cationic transition metal M′ atom center.

Regarding the M′-O bonds of M′≡(O)₃, in embodiments, these three oxygen atoms include those which are also bound to the metal (M″) of the porous metal silicate support as O-M″. This is by contrast to those oxygen atoms of the porous metal silicate support which are bound to the silicon (Si) as O—Si. Thus, in such embodiments, M′ is covalently bound to the porous metal silicate support via one or more (i.e., one, two, or three) M′-O-M″ linkages. Confirmation of the M′-O-M″ linkages may be determined using EXAFS as described in the Example, below. In particular, as shown in FIG. 1E, the existence of the a peak confirms the existence of M′-O-M″ linkages.

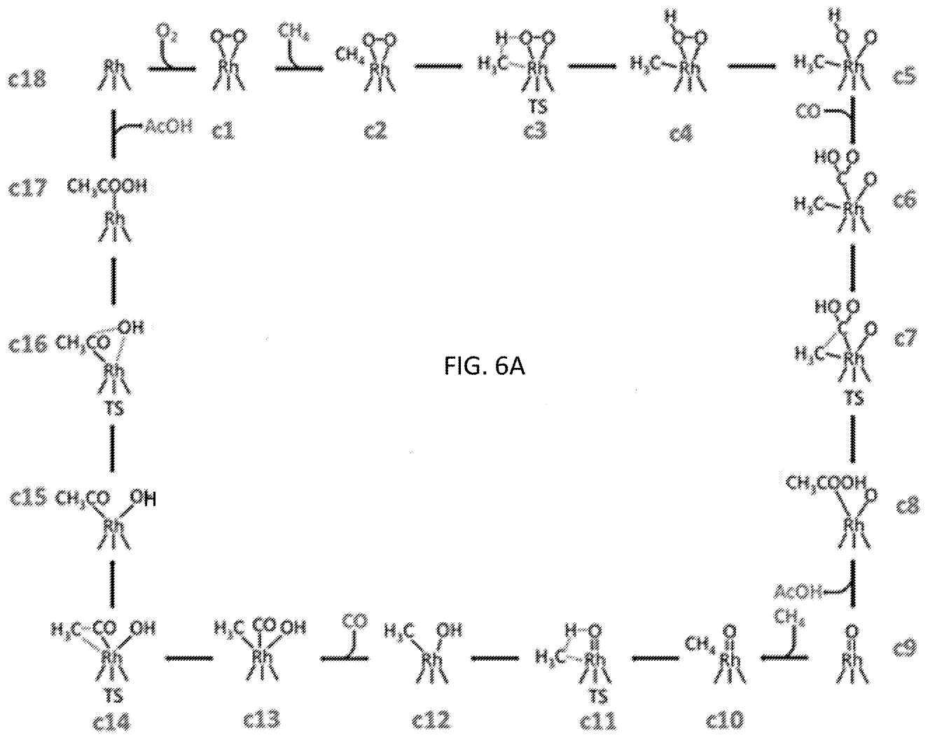

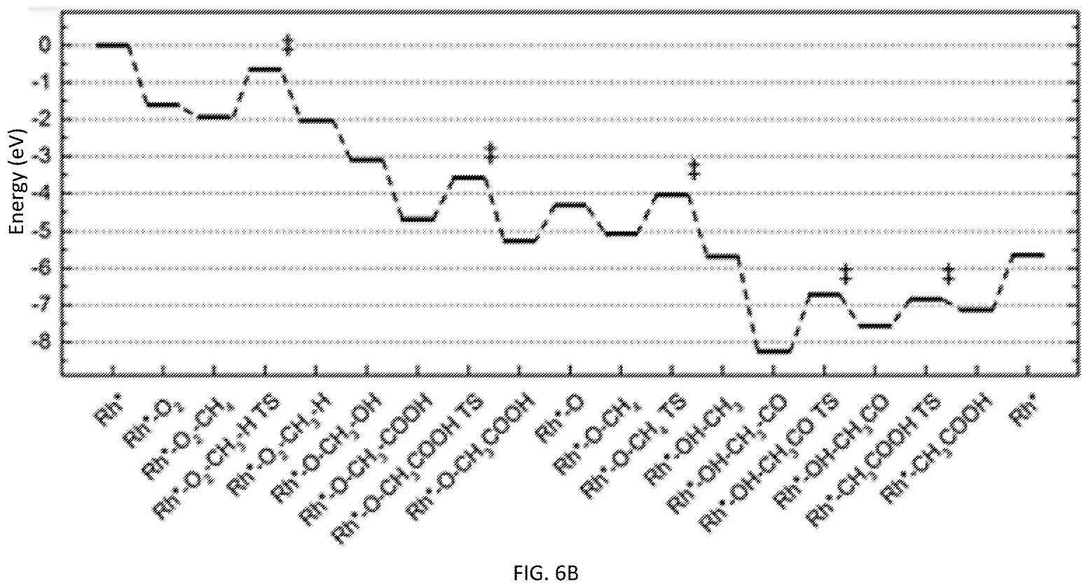

It is believed that the unique structure of the active site of FIG. 1I, involving a CN of 5, the O₂ bridge, and the one or more M′-O-M″ linkages enables the activation of the CH bond of methane and the coupling of the activated methane with carbon monoxide. (See FIG. 6A.) The unique method of making catalysts having such active sites is further described below.

The isolated nature of the cationic transition metal M′ atoms, as well as the active site structure also means that the catalyst may be described as being free of transition metal atoms on an external surface of the porous metal silicate support (versus the internal surfaces of the pores of the porous metal silicate support). In addition, the catalyst may be described as being free of M′-M′ bonds, being free of M′-O-M′ bonds, and/or being free of M′ oxide particles. Confirmation that such species are not present may be achieved using X-ray photoelectron spectroscopy (XPS), inductively coupled plasma atomic emission spectroscopy (ICP-AES), and EXAFS as described in the Example, below. (See also, FIG. 1E-1H).

The catalyst may comprise various amounts of the isolated, cationic, transition metal M′ atoms. The amount may be tuned to ensure isolation of the M′ (i.e., prevent the formation of M′-M′ bonds), as well as tuned to achieve a desired activity and/or selectivity. In embodiments, the amount of M′ in the catalyst is in a range of from 0.01 weight (wt) % to 0.5 weight (wt) %.

In another aspect, methods of making the catalysts are provided. In embodiments, such a method comprises adding a transition metal precursor to a porous metal silicate support comprising hydroxyl Brønsted acid sites under vacuum conditions. The temperature may be room temperature and the addition may continue until the desired amount of transition metal precursor has been added. The porous metal silicate support may be actively mixed during the addition of the transition metal precursor. This step distributes the transition metal precursor throughout the porous metal silicate support so as to fully impregnate the support with the precursor. In addition, the protons of the hydroxyl Brønsted acid sites are exchanged for transition metal atoms of the transition metal precursor. The transition metal precursor is a transition metal-containing compound, e.g., a transition metal salt. The transition metal precursor may be provided as a solution, e.g., as an aqueous solution. Prior to adding the transition metal precursor, the porous metal silicate support may be held under vacuum conditions for a period of time, e.g., from 2 hrs to 6 hrs.

The porous metal silicate support comprising hydroxyl Brønsted acid sites may be formed from a porous metal silicate support precursor comprising other functional groups, e.g., NH₄ groups. The hydroxyl Brønsted acid sites may be achieved by calcining an NH₄ saturated porous metal silicate support precursor in air at an elevated temperature for a period of time. These conditions may be adjusted to provide a desired amount of hydroxyl Brønsted acid sites in the porous metal silicate support, e.g., so that the majority of, or all of, the Brønsted acid sites are the desired hydroxyl Brønsted acid sites. The elevated temperature may be in a range of from 300° C. to 500° C. The period of time may be in a range of from 10 hrs to 14 hrs.

The method further comprises calcining the transition metal-impregnated porous silicate support in air at an elevated temperature for a period of time. The elevated temperature may be in a range of from 500° C. to 600° C. The period of time may be in a range of from 2 hrs to 4 hrs. Prior to calcination, the transition metal-impregnated porous silicate support may be dried in an oven, e.g., at 70° C. to 90° C. for 2 hrs to 4 hrs.

As noted above, it is believed that together, the steps of the disclosed method achieve the unique active site structure described above.

In another aspect, methods of using the catalysts to convert methane to one or more oxygenated products are provided. These oxygenated products may include acetic acid, formic acid and methanol. In embodiments, such a method comprises exposing any of the catalysts described above to a fluid (e.g., a gas mixture) comprising CH₄, CO, and O₂ at a temperature, a pressure, and for a period of time to convert the CH₄ to the oxygenated product(s). The fluid comprises all three reactants (CH₄, CO, and O₂), which are involved in the direct and concerted conversion of methane as described above. (See FIG. 6A.) The conditions, i.e., the temperature, pressure (including reactant partial pressures) and period of time may be adjusted to achieve a desired activity and/or selectivity. The temperature may be no more than 200° C., no more than 175° C., or no more than 150° C. The temperature may be in a range from room temperature (i.e., 20° C. to 25° C.) to 200° C. or from 50° C. to 150° C. The pressure refers to the overall pressure of the reactants, although the partial pressure of each reactant may vary. The overall pressure may be in a range from 5 bar to 100 bar and the reactant partial pressures may each be in a range of from 2 bar to 50 bar. The relative partial pressures may also vary, although generally more CH₄ is present than CO and O₂. By way of illustration, an illustrative ratio of reactants is about 5:1:1. The period of time may be in a range of from 1 hrs to 15 hrs. This period of time refers to the time at the selected temperature.

In the methods of using the catalysts, the catalyst may be provided as an aqueous solution comprising water or as a solution comprising (or consisting essentially of, or consisting of) a hydrophobic solvent. A hydrophobic solvent such as dodecane may be used. This is useful because it facilitates recovery of the oxygenated products (which are hydrophilic) from the reaction media. In such hydrophobic reaction media, the oxygenated products are separated in situ, avoiding the need for additional processing to separate the oxygenated products from water.

The catalysts and the methods of using the catalysts may be characterized by their activity and/or selectivity. Activity may be referenced as a turnover rate (TOR), which may be calculated as described in the Example below. TORs may be calculated for the total amount of oxygenated products produced as well as for a particular oxygenated product. Selectivity for a particular oxygenated product is given as (TOR for the particular oxygenated product)/(total TOR). Activity and selectivity may be referenced with respect to a particular set of conditions, e.g., either condition listed in Table 1. Comparative activities and selectivities may be reported with respect to free transition metal atoms. That is, the same method may be carried out using the same conditions but using free, cationic transition metal M′ atoms (e.g., transition metal M′ cations dissolved in an aqueous solution) instead of the disclosed catalysts. This comparative test is described in the Example, below. In embodiments, the present catalysts exhibit an activity for producing acetic acid that is at least 1000 times greater than that of free, cationic transition metal M′ atoms. Under the same conditions, the present catalysts may exhibit a selectivity for acetic acid of at least 70%.

### EXAMPLE

### Introduction

Catalytic transformation of CH₄under a mild condition is significant for efficient utilization of shale gas under the circumstance of switching raw materials of chemical industries to shale gas. Here, the transformation of CH₄to acetic acid and methanol through coupling of CH₄, CO and O₂ on single site Rh₁O₅ anchored in microporous aluminosilicates in solution at 150° C. is reported. The activity of these singly dispersed precious metal sites for production of organic oxygenates can reach about 0.10 acetic acid molecules on a Rh₁O₅ site per second at 150° C. with a selectivity of 70% for production of acetic acid. It is higher than the activity of free Rh cations by >1000 times. Computational studies suggest that the first C—H of CH₄ is activated by Rh₁O₅ anchored on wall of the micropores; the formed CH₃ couples with CO and OH, forming acetic acid through a low activation barrier.

### Methods

Preparation and characterization of catalyst. Two steps were involved in the preparation of a Rh/ZSM₅ catalyst. The first step was the preparation of H-ZSM₅ by calcining zeolite NH-ZSM5 with a SiO₂/Al₂O₃ ratio of 23 (Alfa) in air at 400° C. for 12 h. Four Rh/ZSM-5 catalysts with different Rh concentrations (0.01 wt %, 0.05 wt %, 0.10 wt %, 0.50 wt %) were synthesized through a method integrating vacuum pumping and incipient wetness impregnation of aqueous solution containing a certain amount of rhodium(III) nitrate hydrate (˜36% Rh basis, Sigma-Aldrich) at room temperature. Typically, 500 mg of H-ZSM-5 was placed in a 50 ml three-port flask. The three ports were sealed with three corks. One port was connected to a vacuum pump. Before injection of the Rh(NO₃)₃ solution, air in the flask containing 500 mg H-ZSM-5 was purged for 3-5 hrs by a vacuum pump when the H-ZSM-5 powder was being stirred. The size of stirring bar was 3 mm for maximizing the amount of H-ZSM-5 to be stirred. Then, 0.30 ml of 1.0 mg/ml Rh(NO₃)₃ aqueous solution was added to the H-ZSM-5 which had been pumped for 3-5 hrs. The injection needle quickly reached the powder to avoid the dispersion of solution to the wall of the flask since the environment of the flask was in vacuum. In addition, the tip of needle was buried in the middle of H-ZSM-5 powder during injection, minimizing diffusion of solution to the wall of the flask. During the injection, the H-ZSM-5 was continuously stirred.

After the introduction of Rh³⁺, the samples were further dried in an oven at 80° C. for 3 hrs and calcined in air at 550° C. for 3 hrs. Other than 0.10 wt % Rh, catalysts with nominal loadings of Rh cations of 0.01 wt % Rh, 0.05 wt % Rh, and 0.50 wt % Rh were used. Actual Rh contents were determined by inductively coupled plasma optical emission spectrometry (ICP-AES). Transmission electron microscopy (TEM) (FEI, Titan 80-300) was used to characterize the morphology of the catalyst. Extended x-ray absorption fine structure spectroscopy (EXAFS) of Rh K-edge was taken at SSRL. For EXASF studies, the used catalyst of 0.10 wt % Rh/ZSM5 was measured when the catalyst was kept at 150° C. in the flow of pure He. The adsorption fine structure spectra of Rh K-edge were fitted using IFEFFIT package and FEFF6 theory. Reference samples including Rh metal foil and Rh₂O₃ nanoparticles supported on Al₂O₃ were studied with EXAFS. Their r-space spectra of these reference samples were fitted with the same software. X-ray photoelectron spectroscopy was performed using a PHI5000 VersaProbe Spectrometer with monochromated Al Kα as the x-ray source.

Catalytic reactions. Transformation of methane to acetic acid on 0.10 wt % Rh/ZSM-5 was performed in a Parr high pressure reactor (Series 4790, Parr) containing a Teflon liner vessel. 28 mg 0.10 wt % Rh/ZSM-5 was added to 10 ml H₂O in the reactor. After evacuating the air left in reactor by flowing CH₄ (99.9%, Matheson) and purging five times, the system was pressurized with reactant gases in a sequence of CH₄, CO (99.9%, Matheson) and O₂ (99.9%, Matheson) to their desired pressures. The high-pressure reactor was completely sealed and then heated to the desired reaction temperature (typically 150° C.) by placing it in an oil bath. The temperature controller of the VWR heating plate was used to measure the temperature of the solution in the Parr reactor through the thermocouple placed in solution of the Parr reactor and to control the temperature through outputting tunable power to the heating plate. Once the desired catalysis temperature was reached, the solution was vigorously stirred at 1200 rpm and was maintained at the reaction temperature for certain amount of time. After completion of the reaction, the vessel was cooled in an ice bath to a temperature below 10° C. to minimize the loss of volatile products. The solution with liquid products was filtered from the catalyst powder. The clean liquid containing acetic acid, formic acid, and methanol was analyzed by ¹H-NMR or ¹³C-NMR. The concentration of Rh in the filtered powder was examined with ICP-AES.

Measurements of products with NMR and GC. ¹H NMR spectra were collected at room temperature on a Bruker AVANCE III HD 400 spectrometer at University of Kansas. The measurement was calibrated by using 3-(trimethylsilyl)-1-propanesulfonic acid sodium salt (DSS) residual signal at δ=0.0 ppm. NMR spectra of products formed from CH₄ transformation were obtained (data not shown). The peak of DSS was identified. Typically, 0.7 ml collected filtrate and 0.1 ml of D₂O (with 0.02 wt % DSS) were mixed in an NMR tube for analysis. The identified oxygenated products were acetic acid (δ=2.05 ppm), formic acid (δ=8.24 ppm), and methanol (δ=3.34 ppm). A solvent suppression program was applied in order to minimize the signal originating from H₂O. To quantify the products, standard curves were built using the method described previously. (Huang, W. X. et al., Angewe. Chem. Int. Ed. 55, 13441-13445 (2016).) To establish a standard curve of a specific product such as acetic acid, a series of standard solutions with different concentrations of acetic acid were prepared. For instance, to establish a standard curve acetic acid, a series of standard solutions with different concentrations of acetic acid were prepared. NMR spectra of these standard solutions were collected with the exact same parameters of NMR measurements. The ratio of the area of peak of acetic acid (δ=2.08 ppm) to area of DSS of the same solution was calculated. These ratios of solutions with different concentrations of acetic acid were plotted as a function of the concentrations of acetic acid, providing a standard curve of acetic acid, formic acid, and methanol (data not shown). Concentration of a product (such as acetic acid) in a solution after catalysis in the Parr reactor was determined by locating the ratio of the peak area of the product to the area of DSS on the Y-axis of the standard curve and then finding the corresponding value on the X-axis, which was the amount of the product of the solution after a catalysis in the unit of μmol. Gases in the head of the Parr reactor after catalysis were analyzed with GC. The amounts of all reactants before catalysis and all products and left reactants after the catalysis were determined (data not shown). This catalysis was performed on 28 mg 0.10 wt % Rh/ZSM-5 dispersed in 10 ml deionized H₂O under 50 bar CH₄, 10 bar CO, and 8 bar O₂ for 2 hrs. ¹H MAS NMR spectra were acquired on a Bruker AVIII 400 MHz spectrometer with a two channel MAS probe (Bruker, Billerica, Mass.). Bruker Topspin 3.5 software was used to acquire, process, and visualize the data.

ICP-AES measurements of concentrations of Rh in catalysts. ICP-AES was used in the measurements of Rh concentration in catalysts before and after catalysis. Four standard solutions with different concentration of Rh³⁺ (0.1 ppm, 1 ppm, 5 ppm, 10 ppm) were prepared by dissolving Rh(NO₃)₃ into de-ionized water. The volume of each solution was 40 ml. The standard curve was built through measuring the four solutions under the exact same setup and parameters of the ICP-AES (mode: JY 2000 2 manufacture: HORIBA) and then plotting the known concentrations of the four solutions as a function of the optical emission spectrometry intensity. Plots of the known concentration of the solution as a function of the optical emission spectrometry intensity were obtained (data not shown). These were standard curves used for the measurements of concentration of Rh in the fresh and used catalysts.

To prepare a test solution of ICP-AES analysis, a certain amount of fresh or used catalyst (0.10 wt % Rh/ZSM-5) was dissolved in NaOH solution through simply mixing the accurately weighed catalyst into a 10 mL 1M NaOH solution and then sonicating the mixture for about 1 hr. Then, aqua regia (a mixture of nitric acid and hydrochloric acid) was added to the solution until the pH was less than 5. The transparent solution was diluted by adding DI water to make the volume of the diluted solution 30 ml. All test solutions were tested under the under the exact same setup and parameters of the same ICP-AES.

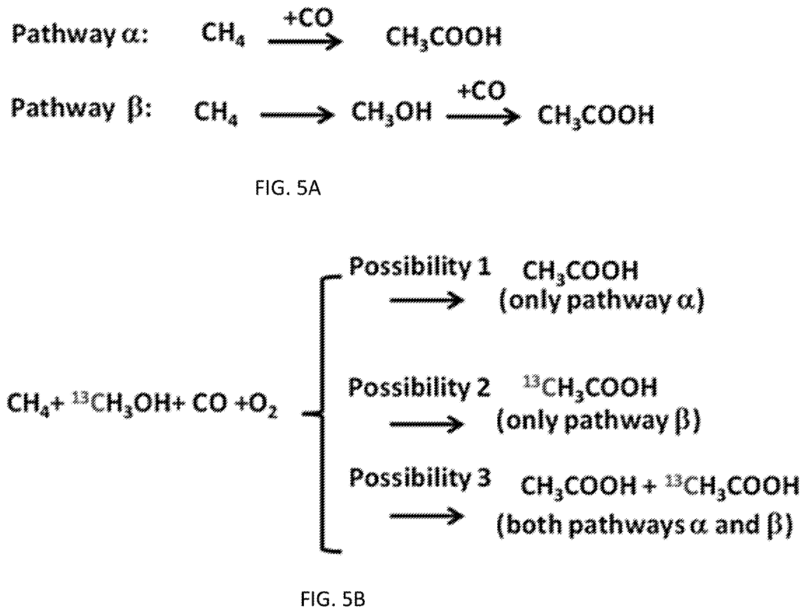

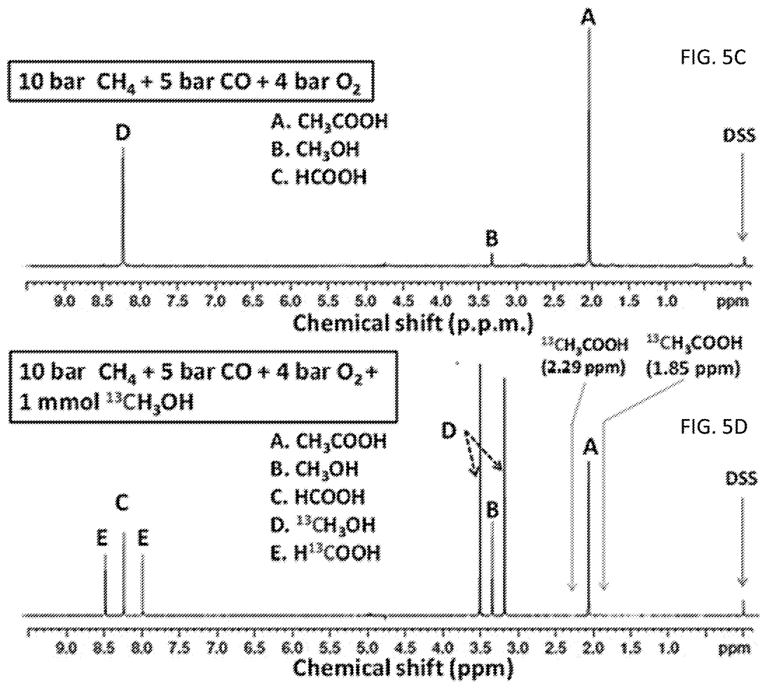

Isotope-labelled experiments using ¹³CH₃OH. To test whether acetic acid could form from carbonylation of CH₃OH on the catalyst 0.10 wt % Rh/ZSM-5, 1.0 mmol isotope-labeled ¹³CH₃OH (99 atom % ¹³C, Aldrich) was added to 10 ml deionized H₂O before introduction of 10 bar CH₄, 5 bar CO, and 4 bar O₂ to the Parr reactor. The purpose of adding an isotope-labeled ¹³CH₃OH to H₂O before catalysis was to test whether ¹³CH₃OH could act as an intermediate to react with CO on the catalyst to form isotope-labelled acetic acid, ¹³CH₃COOH. In reaction pathway β, acetic acid was formed through carboxylation of methanol (FIG. 5A); methanol should be involved definitely. The reaction pathway α in FIG. 5A does not involve CH₃OH. FIG. 5B presents the three sets of possible products if ¹³CH₃OH was added as a probe agent. FIGS. 5C and 5D are a regular ¹H NMR spectrum of solution obtained from 10 bar CH₄, 5 bar CO, and 4 bar O₂ and the ¹H NMR spectrum of solution obtained from 10 bar CH₄, 5 bar CO, and 4 bar O₂ with added 1 mmol ¹³CH₃OH, respectively.

Calculations of TOR of 0.10wt % Rh/ZSM-5. Catalytic activities were calculated in terms of the number of product molecules per Rh site per second. They are listed in Table 1. The following paragraphs describe how they were calculated.

For the catalyst, Rh₁O₅@ZMS-5 (0.10wt % Rh/ZSM-5), 58.7 μmol of acetic acid was formed from 28 mg of catalyst at 150° C. per hr. The concentration of rhodium in the catalyst was 0.10 wt %. The amount of all Rh atoms was:

$$\frac{28 \times {10}^{- 3} gram \times 0.10 \%}{104 gram per mol Rh} = 2.8 \times {10}^{- 7} mol$$

By assuming all Rh atoms anchored to ZSM-5 participated in this catalysis, TOR for production of acetic acid was calculated as the following:

$$T O R = \frac{58 .68 \times {10}^{- 6} mol \times {N}_{A}}{2.8 \times {10}^{- 7} \times {N}_{A} \times 3600 second} = 0.061 acetic molecules per Rh site per second$$

Meanwhile, 14.0 μmol of CH₃OH and 153.4 μmol of formic acid were produced from the same catalyst in one hr. With the same calculation method of TOR, TORs of acetic acid and organic oxygenates under catalysis condition 1 (mixture of 50 bar CH₄, 10 bar CO, and 8 bar O₂ for 2 hrs) and condition 2 (mixture of 50 bar CH₄, 10 bar CO, and 8 bar O₂ for 12 hrs) were calculated and listed in entries 1 and 2 of Table 1.

Calculations of TOR of Rh cations without any support in aqueous solution. A similar experiment was performed. 5 ml of 0.01 mol/l Rh(NO₃)₃ was added in the Parr reactor, and then 50 bar CH₄, 10 bar CO, and 8 bar O₂ were introduced to the Parr reactor. The reaction was performed at 150° C. for about 90 hrs. With the same calculation method, the TORs were calculated. The TORs of all organic products (CH₃COOH, CH₃OH, and HCOOH) and the TOR of acetic acid were 2.4×10⁻⁵ organic molecules and 6.3×10⁻⁶ acetic acid molecules per Rh site per second generated from homogeneous catalyst Rh(NO₃)₃ without any promoter. They are listed in entry 3 of Table 1.

Methods of DFT calculations. The periodic density functional theory (DFT) calculations were performed using the Vienna ab initio Simulation Package (VASP). The Perdew-Burke-Ernzerhof (PBE) functional of generalized-gradient approximation (GGA) was used for the electron exchange and correlation. The D3 method for van der Waals correction by Grimme was used. The electron-core interaction was described using the projector-augmented wave method (PAW). The kinetic energy cutoff was set to 450 eV for the plane wave basis set, and the Brillouin zone was sampled using the gamma point only. A section of a relaxed ZSM-5 framework was used in a cluster model and the dangling bonds capped with hydrogen. The ZSM-5 cluster was placed in an 18×18×22 Å³ box. The Rh site and first neighbors were allowed to relax during the subsequent calculations with the rest of the cluster fixed. The adsorption energies were calculated using E_{ads}=E_{cluster+adsorbate}−(E_{cluster}+E_{adsorbate}), where the energy of the adsorbate E_{adsorbate} was computed by placing the adsorbate in a 15 Å wide cubic cell. Transition states were found using the climbing image nudged elastic band method implemented in VASP, using eight images and a force convergence criterion of 0.05 eV Å⁻¹.

Ready separation of products from solvent by using dodecane. Transformation of CH₄, CO, and O₂ to organic oxygenates was performed at 150° C. for 2 hrs on 28 mg of catalyst while the solvent dodecane was used. The yields of acetic acid and formic acid in dodecane under a mixture of 30 bar CH₄, 10 bar CO, and 5 bar O₂ at 150° C. for 4 hrs were 225 μmol and 82 μmol, respectively (data not shown). The advantage of using dodecane was the ready separation of hydrophilic product molecules from hydrophobic solvent molecules.

### Results

Preparation of isolated Rh catalytic site in ZSM-5. Rh cations were introduced to the internal surface of micropores of ZSM-5 through a method integrating vacuum pumping and incipient wetness impregnation (IWI). To minimize the number of Rh cations to be deposited on the external surface of a ZSM-5 particle, a solution of Rh³⁺ with the same volume as the pore volume of ZSM-5 was slowly dropped to ZSM-5 powder with a syringe pump when the catalyst powder was continuously stirred and remained in vacuum. During IWI, Rh cations exchanged with singly dispersed BrØnsted acid sites of H-ZSM-5, which was prepared through calcining NR-ZSM-5 at 450° C. for 3 hrs. After the introduction of Rh³⁺, the samples were further dried in an oven at 80° C. for 3 hrs and calcined in air at 550° C. for 3 hrs, forming the catalyst, Rh/ZSM-5. The concentration of Rh cations in the as-synthesized catalyst was measured through inductively coupled plasma atomic emission spectroscopy (ICP-AES). Before an ICP-AES measurement, 28 mg of 0.10 wt % Rh/ZSM-5 was dissolved in aqua regia. For a catalyst with a nominal mass ratio of Rh to aluminosilicate, 0.10 wt %, the measured weight percent was 0.10 wt %, which suggests no obvious loss of Rh atoms during the preparation. X-ray photoelectron spectroscopy (XPS) studies of the as-synthesized 0.10 wt % Rh/ZSM-5 show the lack of Rh atoms in the surface region of catalyst particles (FIG. 1C). The lack of Rh atoms in the surface region, revealed with XPS together with the measured 0.10 wt % Rh in the as-synthesized catalyst shown clearly by ICP-AES, suggests that these introduced Rh atoms were anchored in micropores of ZSM-5 particles instead of the external surface of ZSM-5 particles. At a high loading (0.50 wt % Rh/ZSM-5), unfortunately, rhodium oxide nanoparticles (2-4 nm) were formed as low contrast spots in a Transmission Electron Microscopy (TEM) image (FIG. 1B), consistent with the observed Rh 3 d photoemission feature in studies of sample using XPS (FIG. 1C).

The existence of Rh atoms in the micropores of ZSM-5 after catalysis was confirmed by the measured concentration of Rh atoms that remained in micropores with ICP-AES, which was 0.098%. Extended X-ray absorption fine structure spectroscopy (EXAFS) was used to characterize the chemical environment of anchored Rh atoms of used 0.10 wt % Rh/ZSM-5 (the catalyst after reaction). After catalysis, the used catalyst powder was centrifuged and thus washed with deionized H₂O several times and then dried in oven at 200° C. The obtained powder was used for EXAFS studies in flowing He at 150° C. r-space spectrum of the K edge of Rh atoms of the used catalyst show that Rh atoms bond with oxygen atoms, and the average coordination number of oxygen atoms to a Rh atom is CN(Rh—O) of 5.23±0.52 (FIGS. 1E and 1F). Notably, no contribution of Rh—Rh metal bonds was needed to fit the r-space spectrum of Rh K-edge (FIG. 1E), suggesting that there is no evidence for formation of Rh—Rh metal bonds. This is consistent with the oxidization state of Rh shown in FIG. 1D. The second shell of rhodium oxide at 2.60 Å in r-space spectrum (FIG. 1H) was clearly observed in the reference sample of Rh₂O₃ nanoparticles. However, there was a lack of Rh—O—Rh peak at 2.60 Å in the r-space spectrum of 0.10 wt % Rh/ZSM-5 (black line in FIG. 1E). It shows Rh atoms of the used catalyst did not have the second coordination shell of Rh atoms in terms of lack of Rh—O—Rh, and thus there were no rhodium oxide nanoclusters formed in the used catalyst (0.10 wt % Rh/ZSM-5). As shown in FIG. 1E, the peak at about 2.7 Å (α) in r-space spectrum of the Rh K-edge was fit to Rh—O—Al. In addition, the peak at about 3.3 Å (β) in r-space of the Rh K-edge was fit to Rh—O—Si (FIG. 1E); the coordination numbers of Al to Rh and Si to Rh were 1.55±0.66 and 1.68±0.71, respectively; the distances of Al and Si atoms to the Rh atoms were 3.168±0.032 Å and 3.514±0.072 Å, respectively. Thus, these EXAFS studies show that Rh atoms of 0.10 wt % Rh/ZSM-5 are singly dispersed in micropores of ZSM-5 and each Rh atom bond with about five oxygen atoms on average. In the following paragraphs, Rh₃O₅@ZSM-5 was sometimes used when pointing out the coordination environment of the Rh atoms on average. FIG. 11 schematically shows the structure of a catalytic site of Rh₃O₅ anchored in micropores of ZSM-5.

The replacement of Brønsted acid sites (BAS) of H-ZSM-5 by Rh cations was confirmed with ¹H NMR of 0.10 wt % Rh/ZSM-5 and H-ZSM-5.

Catalytic performance of Rh₁O₅@ZSM-5 at 150° C. Catalytic activities of pure H-ZSM-5 and as-prepared Rh/ZSM-5 catalysts were measured by adding 28 mg catalyst to 10 ml deionized water in a Parr high pressure reactor. The aqueous solution with dispersed catalyst particles was continuously and vigorously stirred by a magnetic bar coated with plastic materials at a speed of 600 rpm during catalysis. A mixture of CH₄, CO, and O₂ with different partial pressures was introduced to the Parr reactor at room temperature. A portion of these reactant gases with a relatively high pressure can diffuse to micropores of catalyst dispersed in the solvent (H₂O or dodecane) and thus be catalyzed. Then, the reactor was heated to a set temperature. The reaction temperature of the solvent was directly measured through a thermocouple probe submerged to the solution containing the dispersed catalyst particles and solvent in the Parr reactor. The preservation of catalysis temperature for certain amount of time was performed by a temperature controller. This chemical transformation was performed for certain amount of time. The pressure, reaction temperature, and reaction time of each measurement of catalytic performance are given in the following figures and tables. Catalytic reaction under each condition was repeated at least four times.

After each catalytic reaction, the solution in the Parr reactor consisting of the used catalyst powder and liquid products was filtrated to separate the used catalyst powder. The clear liquid after filtering the catalyst powder mainly contained acetic acid, methanol, formic acid, and solvent. The product solution was analyzed by ¹H NMR and ¹³C NMR. The measurement was calibrated with 3-(trimethylsilyl)-1-propanesulfonic acid sodium salt (DSS) with chemical shift at δ=0.0 ppm. A DSS solution was prepared by dissolving DSS to D₂O, making a solution with concentration of DSS in D₂O at 0.020 wt %. Typically, 0.70 ml of the obtained clear liquid solution was mixed with 0.10 ml of as-prepared DSS solution in an NMR tube before NMR analysis. The identified oxygenate products were acetic acid (δ=2.08 ppm), formic acid (δ=8.28 ppm), and methanol (δ=3.33 ppm). A solvent suppression program was applied for minimizing the signal originating from H₂O. To quantify the amounts of products, standard curves of acetic acid, formic acid, and methanol were carefully established (data not shown).

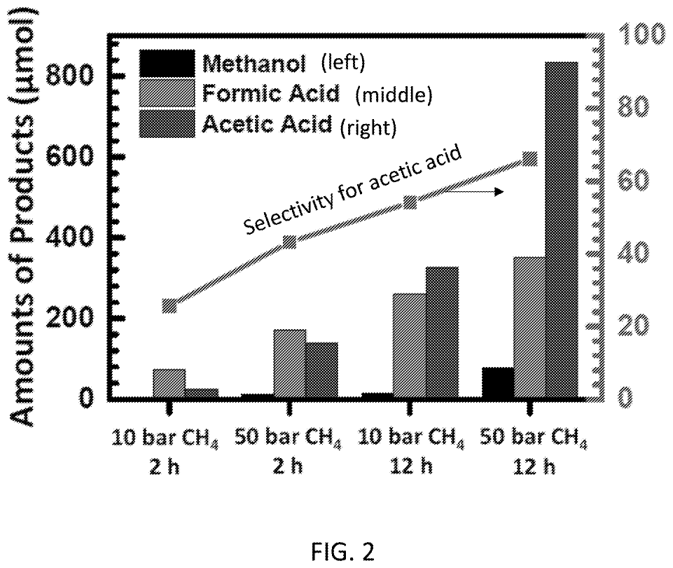

With 28 mg of 0.10 wt % Rh/ZSM-5, 246.0±7.4 μmol of total products (acetic acid, formic acid, and methanol) were produced at 150° C. in the first hr. Under the same condition, the yields of the total organic compounds formed from 0.50 wt % Rh/ZSM-5 were similar to 0.10 wt % Rh/ZSM-5 (data not shown). The similarity in catalytic performances of the two catalysts shows that the rhodium oxide nanoparticles formed on the surface of 0.50 wt % Rh/ZSM-5 are not active for this transformation. The catalytic activity of 0.10 wt % Rh/ZSM-5 in the production of acetic acid is due to the Rh₁O₅ sites anchored in micropores of ZSM-5 instead of rhodium oxide nanoparticles supported on the external surface of ZSM-5. Because the conversions of CH₄ in these studies of FIG. 2 and Table 1 are lower than 20%, these conversions and yields were used to calculate the turn-over rates with the equation:

$$TOR = \frac{number of product molecules}{\begin{matrix}time of catalytic reaction ( S ) \times \\ number of activate sites ( {Rh}_{1} {O}_{5} )\end{matrix}} \qquad ( eq . 1 )$$

This calculation is based on an assumption that all loaded Rh atoms are active sites. The activities for production of acetic acid and total organic oxygenates including acetic acid, methanol, and formic acid at 150° C. are 0.070 and 0.10 molecules per Rh atom per second (entry 2 of Table 1), respectively.

TABLE 1
Comparison of TOR for formation of acetic acid or oxygenates including
acetic acid, formic acid and methanol on 0.10 wt % Rh/ZSM-5.

| | | | | TOR of organic | |
|---|---|---|---|---|---|
| | | | TOR of acetic acid | oxygenate[a] (number | Selectivity |
| | | Catalytic | (molecule per site per | of molecules per site | for production of |
| Entry | Catalyst | temperature | second) | per second) | acetic acid |
| 1 | 0.10 wt % Rh/ZSM-5 [a] | 150° C. | 0.040^{[b]} | 0.099^{[b]} | 40.0% |
| 2 | 0.10 wt % Rh/ZSM-5 [a] | 150° C. | 0.070^{[c]} | 0.10^{[c]} | 70.1% |
| 3 | Rh(NO₃)₃ [a] | 150° C. | 6.3 × 10⁻⁶ ^{[d]} | 2.4 × 10⁻⁵ ^{[d]} | 26.3% |

[a] Here the organic oxygenates include acetic acid, formic acid, and methanol (CO₂ was not included).
^{[b]}The catalysis condition of 0.10 wt % Rh/ZSM-5: mixture of 50 bar CH₄, 10 bar CO, 8 bar O₂, 2 hrs; the yields of acetic acid and all organic oxygenates were listed in FIG. 2.
^{[c]}The catalysis condition of 0.10 wt % Rh/ZSM-5: mixture of 50 bar CH₄, 10 bar CO, 8 bar O₂, 12 hrs; the yields of acetic acid and all organic oxygenates were listed in FIG. 2.
^{[d]} Rh(NO₃)₃ was used. (Lin, M. et al., Nature 368, 613-615 (1994).) 5 ml of 0.01 mol/l Rh(NO₃)₃ was added in the Parr reactor and 50 bar CH₄, 10 bar CO, and 8 bar O₂ were introduced to the Parr reactor and then the Parr reaction was sealed; the reaction was performed at 150° C. for about 90 hrs. This measurement was done for comparison with 0.10 wt % Rh/ZSM-5 in which Rh₁ cations were anchored in micropores.

To check whether Rh atoms anchored in micropores of ZSM-5 could detach from ZSM-5, the clear solution was obtained by filtration for removal of Rh/ZSM-5 catalyst particles from the solution after catalysis at 150° C. for 12 hr. An ICP-AES test of this solution showed only 2% of the total Rh atoms of 28 mg of 0.10 wt % Rh/ZSM-5 detached from ZSM-5 to solution. Thus, most Rh atoms remained in ZSM-5 after catalysis. Due to the negligible amount of Rh³⁺ detached from 0.10 wt % Rh/ZSM-5 and the extremely low TOF of free Rh³⁺ in solution evidenced in entry 3 in Table 1, contribution of the detached Rh³⁺ to the measured catalytic activity in formation of acetic acid was negligible. It suggests that the anchored Rh atoms were the active sites.

To further confirm the contribution of Rh₃O₅ sites to the formation of acetic acid, control experiments were performed on these catalysts including 28 mg of H-ZSM-5, 28 mg of 0.10 wt % Rh/SiO₂, and 28 mg of 0.10 wt % Rh/Al₂O₃ under the exact same condition as that of 28 mg 0.10 wt % Rh/ZSM-5 at 150° C. in the mixture of 10 bar CH₄, 10 bar CO, and 8 bar O₂ for 4 h. As shown in Table 2, the amounts of acetic acid, formic acid, or methanol produced on 28 mg of 0.10 wt % Rh/SiO₂ and 28 mg of 0.10 wt % Rh/Al₂O₃ were lower than 10 μmol, which was at the level of error bar. All the reported yields were the measured products formed from 28 mg catalyst. The yield could be shown as μmol/gram catalyst by multiplying a factor of:

$$\frac{1000 mg \text{/} gram}{28 mg} .$$

For example, the measured yields of methanol and acetic acid on 28 mg of 0.10% Rh/SiO₂ were 8.70 μmol and 6.13 μmol, respectively; if they are multiplied by the factor

$$\frac{1000 mg \text{/} gram}{28 mg} ,$$

they seem to indicate that 310 μmol methanol and 218 μmol acetic acid could form from one gram of 0.10% Rh/SiO₂ catalyst. Here, multiplication was not applied since the values in Table 2 are at the uncertainty level. As the measured 8.70 μmol methanol and 6.13 μmol acetic acid from 28 mg 0.10% Rh/SiO₂ catalyst were in the range of the error bar of these measurements, these values were not used to predict activity of 1 gram to compare with other catalysts. Even if the multiplication factor were applied, the activity of 0.10% Rh/SiO₂ is significantly lower than 0.10 wt % Rh/ZSM-5. For instance, 218 μmol acetic acid from per gram 0.10% Rh/SiO₂ calculated from the measured 6.13 μmol acetic acid per 28 mg is still much lower than 5,000 μmol acetic acid from per gram 0.10 wt % Rh/ZSM-5 calculated from the measured 140 μmol acetic acid per 28 mg catalyst. In conclusion, these control samples in terms of Rh supported on these nonporous oxides and even on a couple of commonly used reducible oxides are not active for the production of acetic acid or methanol from coupling of CH₄ with CO in O₂. Thus, these studies suggest the significant contribution of Rh₁O₅ sites encapsulated in ZSM-5 to the formation of acetic acid.

TABLE 2
Catalytic performances of 28 mg catalysts of 0.10 wt % Rh supported on
different supports in a mixture of 10 bar CH₄, 10 bar CO and 8 bar O₂ at
150° C. for 3 h with 10 ml H₂O in a high-pressure reactor. Acetic acid,
formic acid and methanol were identified as products. Reactants on pure
H-ZSM-5 was also performed at the same conditions as blank
experiment (entry 1).

| | | | Formic | Acetic | Total |
|---|---|---|---|---|---|
| | | Methanol | Acid | Acid | Products |
| Entry | Samples | (μ mol) | (μ mol) | (μ mol) | (μ mol) |
| 1 | H-ZSM5 | 3.67 | 2.28 | 1.87 | 7.82 |
| 2 | 0.10% Rh/SiO₂ | 8.70 | 4.62 | 6.13 | 19.46 |
| 3 | 0.10% Rh/Al₂O₃ | 5.68 | 0.91 | 3.05 | 9.64 |

The participation of all three reactants (CH₄, CO, and O₂) was confirmed with three parallel studies on 0.10 wt % Rh/ZSM-5 under the exact same catalytic condition; in each of these studies, only two of the three reactants were introduced to the Parr reactor; none of these studies produced acetic acid, formic acid, or methanol due to the lack of the third reactant gas. Those studies clearly show that all the three gases (CH₄, CO, and O₂) are necessary reactants for the formation of CH₃COOH. The necessity of the three reactants was supported by DFT calculations described later.

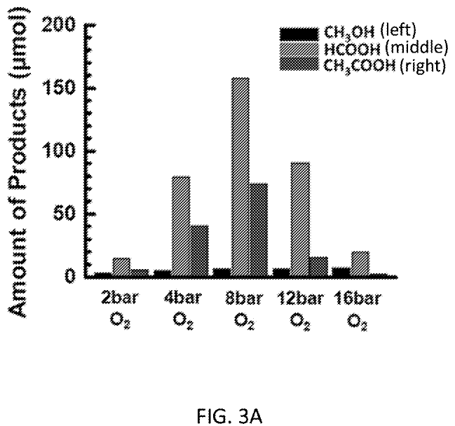

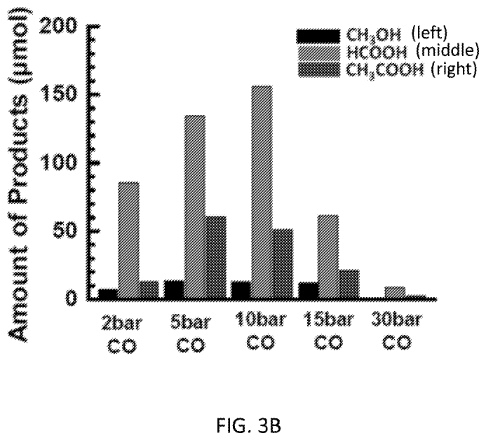

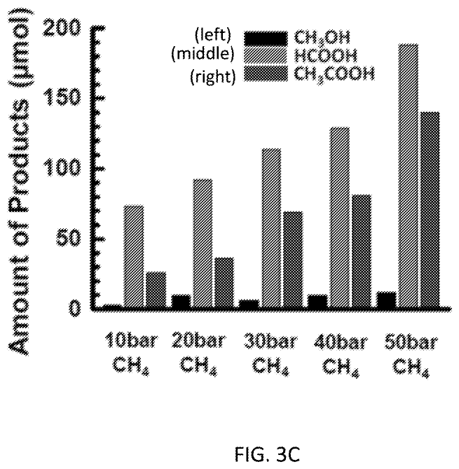

Participation of molecular O₂ in synthesis of acetic acid. Low-cost molecular oxygen (O₂) or compressed air was used as the oxidant in the oxidative transformation of CH₄ and CO to acetic acid. To further confirm the direct participation of molecular O₂, catalysis was performed at different pressures of O₂ (2 bar, 4 bar, 8 bar, 12 bar, and 16 bar), but all other conditions were the same in these parallel studies; in each of these parallel studies, 28 mg of 0.10 wt % Rh/ZSM-5 was added to 10 ml H₂O. The reaction was performed at 150° C. for 2 h in a mixture of 35 bar CH₄, 10 bar CO, and different pressures of O₂, in order to investigate the correlation of yields of products (acetic acid, formic acid, and methanol) with pressure of O₂. As shown in FIG. 3A, the highest yields of acetic acid and formic acid were obtained from the catalysis using 8 bar O₂. The increase of yield of acetic acid and formic acid along with the increase of O₂ pressure shows that O₂ does participate in the formation of acetic acid and formic acid. It is expected that high coverage of oxygen atoms on a Rh atom achieved with a high pressure of O₂ could saturate the coordination of a Rh₁ atom with oxygen atoms and thus poison catalyst sites at a high pressure of O₂, resulting in a low yield of oxygenates at a high pressure of O₂.

Direct participation of CO to synthesis of acetic acid. To further confirm the participation of CO in the formation of acetic acid, influence of the partial pressure of CO on both the conversion of CH₄ and selectivity for production of acetic acid was investigated through parallel studies (FIG. 3B). In each of these studies, the partial pressures of CH₄ and O₂ were fixed at 50 bar and 8 bar, respectively. However, the pressures of CO in the five studies were 2 bar, 5 bar, 10 bar, 15 bar, and 30 bar. The increase of the amount of acetic acid while CO pressure was increased from 2 bar to 10 bar suggests that CO directly participates in the formation of acetic acid. However, the lack of activity for production of acetic acid at 30 bar CO shows that catalyst sites were blocked at such a high pressure of CO. Clearly, CO molecules must have directly interacted with the Rh cations. At a high pressure of CO, high coverage of CO could saturate the coordination of a Rh₁ atom and deactivated this catalyst. The concentrations of Rh were measured in the liquid (α) after filtration of the catalyst experienced the catalysis at 10 bar CO, 50 bar CH₄, and 8 bar O₂ for 2.5 hrs, and in another liquid (β) after filtration of the catalyst experienced the catalysis at 30 bar CO, 50 bar CH₄, and 8 bar O₂. The amounts of Rh atoms in the liquids α and β were 2.0% and 13.0% of all Rh atoms of 28 mg of 0.10 wt % Rh/ZSM-5, respectively. Thus, the much larger loss of Rh atoms at a high pressure of CO (30 bar) suggests that Rh atoms formed carbonyl at a high pressure of CO, and some of these formed rhodium carbonyl species desorbed from micropores and then dissolved in the solution. Thus, at a high pressure of CO, some Rh species detached.

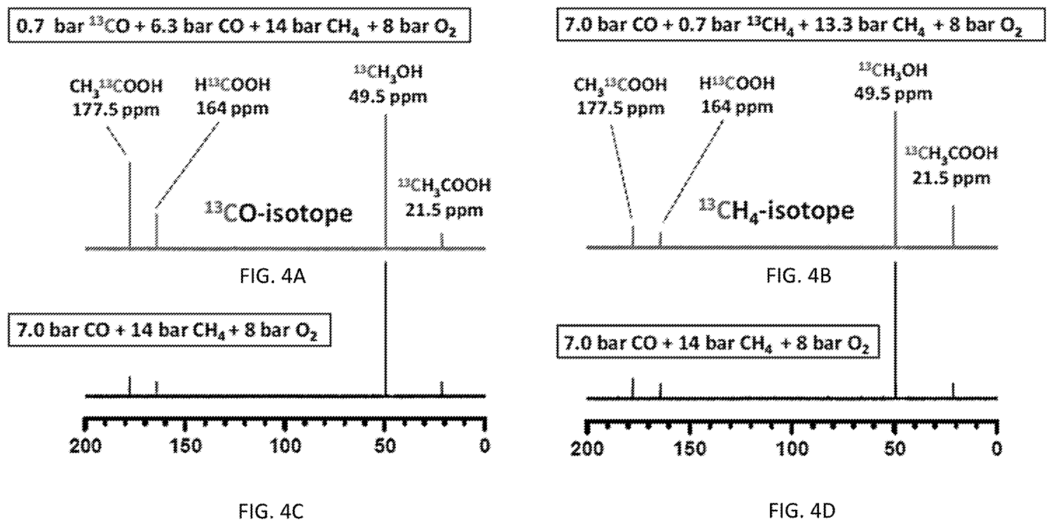

To provide molecular-level evidence on the direct participation of CO in the synthesis of acetic acid, the following isotope experiment was conducted. 0.7 bar ¹³CO (Aldrich, 99%, total pressure 2.5 bar) was mixed with 6.3 bar of CO, 14 bar CH₄, and 4 bar O₂ for catalysis of 10 hrs (FIG. 4A). As the chemical shift of ¹³CH₃OH in ¹³C spectrum can be readily distinguished from acetic acid and formic acid, 40 μmol of ¹³CH₃OH (Aldrich, 99 at %) was added to the collected solution after catalysis as a reference to quantify the amount of potential isotope products ¹³CH₃COOH, CH₃¹³COOH, or H¹³COOH. As the unlabeled CO has a natural abundance of ¹³C of 1.1%, a small amount of CH₃¹³COOH or H¹³COOH can form from the natural 1.10% ¹³CO of the unlabeled CO gas tank. Contrast experiments using the mixture of 7 bar of CO, 14 bar CH₄ and 8 bar O₂ were performed (FIG. 4B). As shown in FIG. 4B, the intensity ratio of the formed CH₃¹³COOH to ¹³CH₃OH in the solution of isotope experiment using ¹³CO (FIG. 4A) was obviously larger than those formed in the contrast experiment using unlabeled CO (FIG. 4B) by 6.4 times; in addition, the intensity ratio of H¹³COOH to ¹³CH₃OH in the isotope experiment (FIG. 4A) was higher than that in the contrast experiment by 2.6 times (FIG. 4B). This suggests that the C′ atoms of CH₃C′OOH and HC′OOH came from C′O molecules. Notably, the intensity ratio of ¹³CH₃COOH to reference (¹³CH₃OH) in the isotope experiment (FIG. 4A) was the same as the ratio of the contrast experiment (FIG. 4B). It suggests that the C atoms of CH₃ of CH₃COOH do not come from the reactant CO. Thus, most ¹³CO molecules were transformed to C′ of CH₃¹³C′OOH and H¹³C′OOH.

One potential pathway to form acetic acid is the coupling of CO with a formed formic acid molecule; if so, yield of acetic acid should increase along the increase of CO pressure. However, as shown in FIG. 3B, the yield of acetic acid decreases along with the increase of CO pressure (10 bar). Thus, coupling formic acid with CO to form acetic acid is not a pathway. To further check the possibility of reaction between HCOOH and CO to form acetic acid, three control experiments were performed at 150° C. for 3 hrs under the following conditions including mixture of 28 mg 0.10 wt % Rh/ZSM-5, 108 μmol HCOOH, and 10 ml DI H₂O without any CO, mixture of 28 mg 0.10 wt % Rh/ZSM-5, 108 μmol HCOOH, and 10 ml DI H₂O with 5 bar CO, and mixture of 28 mg 0.10 wt % Rh/ZSM-5, 108 μmol HCOOH, and 10 ml DI H₂O with 10 bar CO. No acetic acid was formed in these experiments (data not shown).

Direct participation of CH₄ in formation of CH₃COOH. The influence of CH₄ pressure on the catalytic performance was explored at 150° C. under a mixture of 10 bar CO and 8 bar O₂ and different pressures of CH₄ (10 bar, 20 bar, 30 bar, 40 bar, and 50 bar) for 2 hrs (FIG. 3C). The progressive increase of yield of acetic acid shows that CH₄ directly participates in the formation of acetic acid (FIG. 3C), which excludes a pathway in which CH₄ couples with formic acid to form acetic acid. If acetic acid were formed from a coupling of formic acid with CH₄, the amount of formic acid should have decreased along the increase of pressure of CH₄ since more formic acid should have been consumed along with the increased amount of CH₄.

To elucidate the source of carbon atoms at the molecular level, ¹³CH₄ isotope experiments were performed. 0.7 bar ¹³CH₄ (Aldrich, 99 at %) was mixed with 13.3 bar of CH₄, 7.0 bar CO, and 4.0 bar O₂ for isotope experiment on 28 mg of 0.10 wt % Rh/ZSM-5 at 170° C. for 10 hrs (FIG. 4C). A control experiment using 14 bar unlabeled CH₄ was performed under the exact same catalytic condition (FIG. 4D). ¹³CH₃COOH were formed in the two experiments. However, the ratio of ¹³CH COOH to reference (¹³CH OH) when ¹³CH₄ was used (FIG. 4C), was much larger than that when unlabeled CH₄ was used (FIG. 4D). This difference shows that the carbon atom of CH₃ of acetic acid comes from CH₄ instead of CO. If C atoms of C═O of CH₃COOH could come from CH₄, the ratio of CH₃¹³COOH to reference (¹³CH₃OH) in FIG. 4C would be much larger than the ratio in FIG. 4D since the experiment of FIG. 4D contains a significant amount of ¹³CH₄. In fact, in both experiments (FIGS. 4C and 4D), a small amount of CH₃¹³COOH was observed, but there was no difference between their ratios to reference (¹³CH₃OH) in the experiments of both FIGS. 4C and 4D. Here the formation of CH₃¹³COOH is due to the natural abundance of ¹³CO in unlabeled CO. Thus, CO does not contribute to the formation of CH₃ of CH₃COOH.

Direct coupling of reactants for formation of acetic acid. It is noted that the amounts of the observed methanol are always much lower than acetic acid and formic acid. One potential argument for the low yield of methanol could be that methanol has been formed but it could have acted as an intermediate in formation of acetic acid; in other words, it could have been consumed through coupling with CO to form acetic acid. Depending on whether CH₃OH could act as an intermediate product in the formation of acetic acid or not, two categories of potential pathways α and β were proposed FIG. 5A. In potential pathway α, CH₄ couples with CO to directly form acetic acid; methanol is not an intermediate product of this type of reaction pathway. In potential pathway β, however, CH₃OH is an intermediate product and then is consumed in the formation of acetic acid; CH₄ is first oxidized to CH₃OH (the first step) and then CH₃OH couples with CO to form acetic acid (the second step); the second step of the potential pathway β is called carbonylation of methanol by CO; it is in fact the Monsanto process. In order to identify whether CH₃OH carbonylation (pathway β) could be a pathway for the production of acetic acid on the catalyst Rh₁O₅@ZSM-5, carefully designed isotope experiments were performed.

These isotope experiments show that acetic acid cannot be formed from carbonylation of methanol by CO on the catalyst. In one isotope-labeled experiment, 1.0 mmol isotope-labeled ¹³CH₃OH (99 atom % ¹³C, Aldrich) was added to 10 ml deionized H₂O before introduction of 10 bar CH₄, 5 bar CO, and 4 bar O₂ to the Parr reactor. If CH₃OH could not be an intermediate for formation of acetic acid, the added ¹³CH₃OH would not participate in the formation of isotope-labeled acetic acid, ¹³CH₃COOH. Thus, no ¹³CH₃COOH could be observed if carbonylation of methanol by CO would not be involved (possibility 1 in FIG. 5B). The NMR spectrum of the solution of products formed in the reactor having ¹²CH₄, ¹²CO, and O₂ in H₂O (¹³CH₃OH was not added) after a reaction of 2 hrs was presented in FIG. 5C. FIG. 5D is the NMR spectrum of the products formed after the catalysis for 2 hrs under a condition of mixture of ¹³CH₃OH, ¹²CH₄, ¹²CO, and O₂ at 150° C. The observed peaks A, B, C, D, and E in FIG. 5D were assigned to CH₃COOH, CH₃OH, HCOOH, ¹³CH₃OH, and H³COOH, respectively. As neither peak of H atoms of ¹³CH₃ of ¹³CH₃COOH in ¹H spectrum nor peak of ¹³C atoms of CH₃¹³COOH in ¹³C spectrum was observed in the NMR spectrum of FIG. 5D, pathway β is not a pathway for formation of acetic acid. Thus, these isotope studies show that acetic acid is not formed from carbonylation of methanol by CO. Additionally, H¹³COOH was observed clearly in FIG. 5D, suggesting that ¹³CH₃OH can be oxidized to H¹³COOH under the current catalytic condition.

The dry reforming of CH₄ by CO₂ was also performed by introducing 30 bar CH₄ and 30 bar CO₂ to the reactor containing 10 ml H₂O and the well dispersed 28 mg of 0.10 wt % Rh/ZSM-5. The reactor was heated to 150° C. and remained at 150° C. for 5 hrs and then cooled to 10° C. in ice water. NMR test shows none of these products (acetic acid, formic acid, and methanol) was formed (data not shown).

Ready separation of products from hydrophobic solvent. The above chemical transformation was performed in aqueous solution. As the products of this chemical transformation, acetic acid, formic acid, and methanol, are hydrophilic, these hydrophilic products must then be separated from water. To make these hydrophilic products automatically separate from solvent after synthesis, a hydrophobic solvent, n-dodecane, was used. The yields of catalysis at 150° C. on 28 mg 0.10 wt % Rh/ZSM-5 in 10 ml n-dodecane in the mixture of 30 bar CH₄, 10 bar CO, and 5 bar O₂ were calculated, showing the catalyst is definitely active for the formation of acetic acid (data not shown). The significant advantage of using the hydrophobic solvent is that the hydrophilic products of this reaction, including acetic acid, methanol, and formic acid, can be readily separated from the hydrophobic solvent, with or without a low energy cost.

Feature of this mild oxidation of methane in solution. CH₄ and CO can be oxidized with different oxidants including O₂, concentrated H₂SO₄, or a superacid by using a homogeneous catalystin which acetic acid and other products (formic acid and methanol) are formed. One control experiment was done (entry 3 in Table 1); the turn-over-rate (TOR) of the homogenous catalyst, Rh(NO₃)₃, without a promoter is only 6.3×10⁻⁶ molecules per rhodium cation per second at 150° C. By contrast, the Rh₁O₅@ZSM-5 catalyzes the oxidation of CH₄ and CO with a low-cost oxidant, molecular oxygen, or even air at 150° C. at a solid-liquid-gas interface. TOR of the catalytic sites Rh₁O₅ anchored in microporous silicate reached 0.070 CH₃COOH molecules per Rh₁O₅ site per second in a mixture of 50 bar CH₄, 10 bar CO, and 8 bar O₂ (entry 2 of Table 1). These TORs for production of acetic acid on singly dispersed site Rh₁O₅ are higher than reported homogenous catalysts by >1000 times. (Periana, R. A., et al., Science 301, 814-818 (2003); Lin, M et al., 1994) As shown in FIG. 2, 840 μmol of acetic acid, 352 μmol of formic acid, and 82 μmol of methanol were produced from 28 mg 0.10 wt % Rh/ZSM-5 at 150° C. for 12 hrs under a catalytic condition of 50 bar CH₄, 10 bar CO, and 8 bar O₂, which corresponds to conversion of 10.2% of CH₄ under this condition. Selectivity for production of acetic acid among all organic products reaches about 70% under this condition. Other than the highest catalytic efficiency on Rh₁O₅@ZSM-5, a significant advantage of this catalytic process is the ready separation of liquid products from the solid catalyst and solvent.

It is found that short reaction times correlate to a high selectivity for formation of formic acid, and a longer reaction time leads to a higher selectivity for formation of acetic acid. Both formic acid and acetic acid are the main products when reaction time is shorter than 3 hrs (data not shown). When the reaction time is 3 hrs or longer, acetic acid is the main product. The evolution of the yields of formic acid and acetic acid as a function of time implies that the relative low temperature of catalyst in the heating from 25° C. to 150° C. is favorable for the formation of formic acid.

Understanding reaction mechanism at molecular level. Based on the coordination environment of Rh₁ atoms suggested by EXAFS studies, a structural model was used whose Rh atom bonded with three oxygen atoms of the substrate wall and two oxygen atoms of one oxygen molecule in the computational studies. The DFT calculations suggest that the Rh atom prefers a ten-membered-ring channel, which gives smaller repulsion, instead of a six-membered ring channel of ZSM-5. Based on the experimental preparation method, it was expected that the Rh₁ cations would replace the Bronsted site and thus bind to the Al atoms in the Si—O framework. As shown in FIG. 6A this Rh₁ atom binds to three oxygen atoms of the Si—O framework and two oxygen atoms of reactant, making Rh₁ exhibit positive to 0.927 |e|.

Isotope experiments suggest two necessary steps: activation of C—H bond of CH₄ to form CH₃ and insertion of CO to form acetic acid. Based on these suggestions, reaction pathway on the Rh₁O₅ with lowest energy was simulated and transition states were located (FIGS. 6A-6B). The energy profile and catalytic cycle are illustrated in FIGS. 6A-6B. (The specific energies were calculated but are not shown). It was found that the Rh₁O₅ active site (FIG. 6A) participates in the reaction by first activating C—H bond of methane (c2 and c3) with a reaction barrier of 1.29 eV. It forms a methyl and hydroxyl adsorbed on the Rh atom (c4). Then, a CO molecule can insert to the Rh—O bond of Rh—O—H, forming a HOOC adsorbed on Rh. The HOOC can inset into the methyl-Rh bond with a barrier of 1.11 eV, forming a weakly adsorbed acetic acid (c8). A following desorption gives the first CH₃COOH molecule. The remaining Rh—O oxo group (c9) activates C—H bond of the second CH₄ molecule to form a methyl and a hydroxyl group adsorbed on the Rh₁ atom (c12). Following, or concurrently to, this step, the second CO molecule binds to the unsaturated Rh site (c13). Then, the adsorbed CO inserts into the methyl-Rh bond with a barrier of 1.54 eV, forming an acetyl group (c15). Finally, the hydroxyl group couples with carbon atom of C═O of the acetyl to form the second acetic acid with a barrier of 0.72 eV (c17). Desorption of the second acetic acid molecule results in Rh site (c18) which can bond with a molecular O₂, forming a Rh₁O₅ site (c1) ready for the next catalytic cycle.

The experimental studies show that a high pressure of CO (FIG. 3B) in fact decreased the activity for producing acetic acid and finally poisoned the active sites. Computational study explored the observed influence of CO pressure on the catalytic activity. It suggests that saturated coordination of Rh with CO molecules at high pressure can poison a Rh₁ site and thus prevent it from forming acetic acid. In addition, the DFT calculations show the activation barrier for C—H of CH₄ is largely increased if the Rh₁ pre-adsorbed two CO molecules at a high pressure of CO.

### Additional Discussion

Does acetic acid form from reaction of CO with formic acid? To test whether acetic acid could form through reaction between formic acid and CO, three experiments were performed by adding 20 mg 0.10 wt % Rh/ZSM-5 into 10 ml H₂O, dispersing HCOOH into 10 ml DI H₂O and introducing 0 bar CO, 5 bar CO, or 10 bar CO and then heating the solution to 150° C. and keeping it at 150° C. for 3 hrs. There was not any acetic acid formed in the experiments (data not shown). Thus, formation of acetic acid from coupling between formic acid and CO is not a possible pathway for synthesis of acetic acid from CH₄, CO, and O₂.

Does acetic acid form from dry reforming of CH₄? Direct reforming of CH₄ with CO₂ to produce acetic acid at a temperature 250° C. has been reported. Presumably, one potential reaction pathway for the formation of acetic acid on a catalyst is that CO could be first oxidized by O₂ to form CO₂, and then CO₂ could couple with CH₄ to form acetic acid. To check this possibility, 30 bar CH₄ and 30 bar CO₂ were introduced to the Parr reactor, and the reaction was performed under the same catalytic condition on 28 mg 0.10 wt % Rh/ZSM-5 (at 150° C. for 4 hrs). No acetic acid, formic acid, or methanol was formed (data not shown). Thus, the pathway consisting of CO oxidation to form CO₂ and then reforming CH₄ with CO₂ to form acetic acid was excluded.

Preservation of Rh cations in micropores after catalysis. One concern is whether Rh cations were still in the micropores after catalysis. Solution after catalysis consisted of solvent and products (in liquid) and solid catalyst. As most zeolite particles deposited to the bottom, they were readily separated after centrifugation. Notably, small particles couldn't be precipitated; thus, filter paper was used to filter these small catalyst particles from the solution after majority catalyst particles were deposited through centrifugation. In this way, the most solid catalyst particles were collected for ICP analysis.

The collected catalyst (after catalysis) was dissolved in solution for the ICP test. The details of the preparation of the solution were described in the section entitled “ICP-AES measurements of concentrations of Rh in catalysts” above. ICP-AES tests showed that the Rh atoms in the collected catalyst was 0.096 wt % Rh, very close to the original weight ratio of Rh, 0.10 wt % Rh. It suggests that there was little leaching of Rh from ZSM-5. From this point of view, Rh cations remained in the micropores during catalysis.

Do Rh cations chemically bond to O atoms in micropores? A fundamental question is whether Rh cations chemically bond to oxygen atoms in micropores or only physisorb in the micropores. To check the oxidation state and coordination environment of the Rh atoms in micropore after catalysis, XANES and EXAFS studies of the used catalysts were performed. The measured distance between Rh and O atoms from r-space of Rh K-edge (FIG. 1E) was 2.016 Å, which is very close to the Rh—O bond length of Rh₂O₃ reference sample. Thus, Rh cations are definitely anchored on oxygen atoms of micropores. In addition, the observation of peaks α, Rh—(O)—Al and β, Rh—(O)—Si in r-space spectrum of Rh K-edge of 0.10 wt % Rh/ZSM-5 after catalysis (FIG. 1E) further support that Rh atoms anchor on oxygen atoms of the wall of the micropores of ZSM-5.

Why is selectivity high for producing formic acid at short reaction? The catalytic performances in FIG. 2 obtained at 10 bar CH₄, 10 CO, and 8 bar O₂ for 2 hrs and 50 bar CH₄, 10 CO, and 8 bar O₂ for 2 hrs in FIG. 2 and data in FIG. 3 were collected after 1.5 or 2 hrs. The selectivity for formation of formic acid is higher than that for acetic acid. However, a longer reaction time such as the data under the catalytic conditions (in the mixture of 10 bar CH₄ with 10 bar CO and 8 bar O₂ for 12 hrs or the mixture of in 50 bar CH₄ with 10 bar CO and 8 bar O₂ for 12 hrs in FIG. 2) gave selectivity for formation of acetic acid higher than formic acid.

The high selectivity for formic acid (the low selectivity for acetic acid) is relevant to the large portion of incubation heating of catalyst from 25° C. to 150° C. among a whole heating when the formal heating time at 150° C. is short. Here, the whole heating of catalyst includes the incubation heating from 25° C. to ideal temperature (typically 150° C.) and formal heating at the ideal temperature (typically 150° C.); the time reported for heating is only the time of reactor remaining at ideal temperature (typically 150° C.); the time used for heating the catalyst from 25° C. to 150° C. (called incubation heating) is about 1 hr. If the formal heating time at ideal temperature is only 2 or even 1 hr, the incubation heating is an important portion of the whole heating. If the formal heating time at ideal temperature is 12 hrs, the incubation heating is a minor portion of the overall heating.

Since the selectivity for formation of formic acid in heating for a short time is higher than that of heating for a long time, it suggests that a relatively low temperature of incubation heating from 25° C.-150° C. favors the formation of formic acid. When the heating time is only 1 or 2 hrs, the incubation heating from 25° C. to 150° C. probably mainly forms formic acid and thus results in a relatively high selectivity for the formation of formic acid. This interpretation is consistent with the proposed reaction pathway by DFT calculation. As shown in the energy profile FIG. 6B, to form acetic acid, the barrier across the transition state (c7 in FIG. 6A) from c6 to c8 in FIG. 6A to form the first acetic acid is quite high. This high barrier makes the formation of the first acetic acid at low temperature not kinetically favorable. Alternatively, the intermediate (c6 in FIG. 6A), a formate (HCOO) adsorbed in Rh could readily couple with one H to form formic acid at low temperature to desorb from the site, instead of crossing the high barrier of the transition state (c7 in FIG. 6A) to form acetic acid.

To further check whether this interpretation is correct or not, a time-dependent study was performed of the yields of formic acid and acetic acid, respectively. The parallel studies were done for formal heating at 150° C. for 1 hr, 3 hrs, 5 hrs, and 12 hrs under the same condition (30 bar CH₄ 10 bar CO and 8 bar O₂). The selectivity for formation of acetic acid increases as a function of time (data not shown). This is consistent with the kinetically favorable formation of formic acid at low temperature since an experiment with short formal heating time at 150° C. has a large portion of heating at low temperature (25° C.-150° C.). Thus, the understanding of the high selectivity for producing formic acid is supported by the experiments.

Understanding the CO pressure-dependent catalytic activity through computation. The experimental studies found that CO at high pressure (FIG. 3B) in fact decreased the selectivity for producing acetic acid and finally poisoned the active sites. To understand this observation, the CO adsorption on the Rh₁ atom was evaluated in DFT calculations. It was found that the first adsorbed CO molecule binds strongly to the Rh₁ site, with an adsorption energy of −1.92 eV, and −0.69 eV for the second CO. It suggests that the Rh₁ site could adsorb two CO molecules. Thus, the crowd packing of two CO molecules on Rh₁ of the catalyst in liquid under CO gas at high pressure CO largely limits the access of CH₄ molecules. In other words, the Rh₁ site could be readily poisoned by adsorption of two CO molecules under CO gas at high pressure. The saturated binding of a Rh₁ atom with CO molecules at high pressure prevents Rh₁ from activating C—H bond of CH₄.

The C—H activation of methane by a Rh₁ atom when the Rh₁ has already adsorbed a CO molecule was also explored by examining the transition state in activation of the first C—H of CH₄ on Rh₁ with one pre-adsorbed CO molecule. With a pre-adsorbed CO molecule on Rh₁O₅, the barrier for activating the first C—H of CH₄ is only 0.34 eV. Unfortunately, the activation barrier for activating CH₄ on a Rh₁ atom with two pre-adsorbed CO molecules is increased to 1.36 eV. The large increase of activation barrier for activating CH₄ suggested by DFT calculation rationalized the poison of CO to Rh₁O₅ sites in the formation of acetic acid when CO pressure is higher than 10 bar, observed in FIG. 3B.

### Conclusions

In summary, the heterogeneous catalyst, 0.10 wt % Rh/ZSM-5, consisting of singly dispersed Rh₁O₅ sites anchored in the micropores of microporous aluminate silicate, was prepared. The anchored Rh₁O₅ sites exhibited unprecedented catalytic activity in synthesis of acetic acid higher than free Rh³⁺ in aqueous solution by >1000 times. This heterogeneous catalytic process opens a new route to synthesize acetic acid through direct utilization of methane under a mild condition at a low temperature 150° C. by using a low-cost oxidant, O₂ or air, instead of current industrial process of synthesizing acetic acid through carbonylation of methanol.

In this disclosure, additional information, including “data not shown,” may be found in U.S. Patent Application Ser. No. 62/648,105, the entire contents of which are hereby incorporated by reference.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. A catalyst for producing one or more oxygenated products from methane, the catalyst comprising active sites comprising isolated, cationic transition metal M′ atoms covalently bound to internal surfaces of pores of a porous metal M″ silicate,
wherein M′ is Rh or Ir, and
further wherein the M′ atoms are bound to five oxygen (O) atoms.

2. The catalyst of claim 1, wherein the active sites have formula (O₂ )=M′≡(O)₃, wherein O₂ is molecular oxygen and the remaining O atoms are also covalently bound within the porous metal M″ silicate.

3. The catalyst of claim 2, wherein one, two, or all three of the oxygens of the M′≡(O)₃ bonds are also covalently bound to the M″ of the porous metal M″ silicate, thereby providing one, two, or three M′-O-M″ linkages.

4. The catalyst of claim 1, wherein an external surface of the porous metal M″ silicate is free of M′ atoms, the porous metal M″ silicate is free of M′-M′ bonds, the porous metal M″ silicate is free of M′ oxide particles, or combinations thereof.

5. The catalyst of claim 1 having an amount of M′ in a range of from 0.01 wt % to 0.5 wt %.

6. The catalyst of claim 1, wherein the porous metal M″ silicate is a microporous aluminosilicate.

7. The catalyst of claim 6, wherein the microporous aluminosilicate is a zeolite.

8. The catalyst of claim 7, wherein the zeolite is ZSM-5.

9. A catalyst for producing one or more oxygenated products from methane, the catalyst comprising active sites comprising isolated, cationic transition metal M′ atoms covalently bound to internal surfaces of pores of a porous metal M″ silicate,
wherein M′ is Rh or Ir,
wherein the active sites have formula (O₂ )=M′≡(O)₃, wherein O₂ is molecular oxygen and the remaining O atoms are covalently bound within the porous metal M″ silicate, and
further wherein one, two, or all three of the oxygens of the M′≡(O)₃ bonds are also covalently bound to the M″ of the porous metal M″ silicate, thereby providing one, two, or three M′-O-M″ linkages.

10. The catalyst of claim 9, wherein M′ is Rh and the porous metal M″ silicate is a zeolite.

11. The catalyst of claim 10, wherein the zeolite is ZSM-5.

12. A method of making the catalyst of claim 1, the method comprising
adding a transition metal M′ precursor to a porous metal M″ silicate support comprising hydroxyl Brønsted acid sites under vacuum conditions to provide an impregnated porous metal M″ silicate, and
calcining the impregnated porous metal M″ silicate in air at an elevated temperature and for a period of time to provide the catalyst of claim 1.

13. The method of claim 12, further comprising forming the porous metal M″ silicate support comprising hydroxyl Brønsted acid sites by calcining a porous metal M″ silicate support precursor in air at an elevated temperature and for a period of time.

14. The method of claim 13, further comprising drying the impregnated porous metal M″ silicate prior to calcining the impregnated porous metal M″ silicate.

15. A method of using the catalyst of claim 1, the method comprising exposing the catalyst to a fluid comprising CH₄, CO, and O₂ at a temperature, a pressure and for a period of time to convert the CH₄ to the one or more oxygenated products selected from acetic acid, formic acid and methanol.

16. The method of claim 15, the temperature is no more than 200° C.

17. The method of claim 15, wherein the catalyst is provided as a solution comprising a hydrophobic solvent.

18. The method of claim 15, wherein the catalyst exhibits a turnover rate for producing acetic acid of at least about 1000 times greater than that of free, cationic transition metal M′ atoms in solution.

19. The method of claim 18, wherein the catalyst exhibits a selectivity of acetic acid of at least 70%.

20. The method of claim 15, wherein the CH₄ is provided as shale gas.
